(19) United States
(12) Patent Application Publication
(10) Pub. No.: US 2004/0127584 A1
(43) Pub. Date: Jul. 1, 2004

(54) Chemical vapor deposition synthesis of polymerization catalyst composition

(75) Inventor: Lyle R. Kallenbach, Bartlesville, OK (US)

Correspondence Address: Jeffrey R. Anderson, RICHMOND, HITCHCOCK, FISH & DOLLAR, P.O. BOX 2443, Bartlesville, OK, 74005, US

(73) Assignee: Phillips Petroleum Company, Bartlesville, OK

(21) Appl. No.: 10/334,773

(22) Filed: Dec. 31, 2002

(51) Int. Cl. C07C 27/06

(52) U.S. Cl. 518/715; 502/325

(57) ABSTRACT

A catalyst composition and a process of using a catalyst composition for preparing high molecular weight hydrocarbons, such as polymethylene, from a fluid containing hydrogen and carbon monoxide are disclosed. The catalyst composition contains ruthenium and a support component. The ruthenium is initially present as ruthenium tetraoxide provided by contacting a ruthenium component and an oxidizing agent to provide the ruthenium tetraoxide that is deposited under a depositing condition on, in, or with the support component.

### CROSS-REFERENCES TO RELATED APPLICATIONS

This patent application is related to U.S. patent application Ser. No. ______ , entitled “Catalyst Composition Comprising Ruthenium and Zirconium and Processes Therefor and Therewith for Preparing High Molecular Weight Hydrocarbons such as Polymethylene” to L. Kallenbach et al. (Attorney Docket No. 33986US/30272-P004US), filed concurrent herewith and incorporated herein by reference.

### BACKGROUND OF THE INVENTION

The present invention relates to a process of preparing high molecular weight hydrocarbons, particularly polymethylene, in the presence of a catalyst composition.

It is known that reacting a synthesis gas, a mixture of carbon monoxide and hydrogen, at very high pressures, for example greater than 15,000 pounds per square inch gauge (psig), and at temperatures of from about 100° C. to about 500° C. in the presence of supported catalysts can provide high molecular weight hydrocarbons such as polymethylene. However, the pressures required to produce such products from synthesis gas are difficult to achieve, require specific equipment, involve many safety issues, and have a negative impact on the economics of the process. Thus, production of high molecular weight hydrocarbons, such as polymethylene, utilizing moderate reaction conditions that do not require high pressures and related equipment needed to handle the high pressures would be a significant contribution to the art and to the economy.

It is also known that a serious problem associated with synthesis gas operations has been the non-selectivity of the product distribution since high activity catalysts generally yield a liquid product containing numerous hydrocarbon materials such as methanol and branched chain higher alcohols. Complicated recovery schemes are necessary to separate the desired products and the overall yield of the valuable organic products is low. Thus, a process that can produce high molecular weight hydrocarbons, such as polymethylene, from synthesis gas to provide a high degree of selectivity to polymethylene would also be of significant contribution to the art and to the economy.

It is also known that supported ruthenium catalyst compositions have been used at high pressure reaction conditions for producing polymethylene from synthesis gas. However, such catalyst compositions require the use of high pressures and are not useful for obtaining a high yield of polymethylene from synthesis gas conversion at moderate reaction conditions. Further, such supported ruthenium catalyst compositions are typically prepared utilizing impregnation techniques such as solution impregnation or incipient wetness impregnation techniques. Such impregnation techniques require a substantial amount of ruthenium to be utilized in order to impregnate enough ruthenium on the support to provide a catalyst composition that can be utilized for preparing high molecular weight hydrocarbons, such as polymethylene, from a fluid containing carbon monoxide and hydrogen. When impregnation techniques are utilized, typically greater than 10 weight percent ruthenium based on the total weight of the catalyst composition is required to be impregnated in order to obtain a catalyst effective for preparing high molecular weight hydrocarbons such as polymethylene. Thus, a supported ruthenium catalyst composition, a process of making such catalyst composition, and a process for using such catalyst composition for producing polymethylene from synthesis gas that provides a high yield of polymethylene at moderate reaction conditions and utilizes significantly less ruthenium compared to supported ruthenium catalyst compositions prepared using impregnation techniques that are utilized at high pressure conditions would also be of significant contribution to the art and to the economy.

### SUMMARY OF THE INVENTION

It is an object of the present invention to provide a process for contacting, under reaction conditions, a catalyst composition comprising ruthenium and a fluid comprising hydrogen and carbon monoxide to provide high molecular weight hydrocarbons, such as polymethylene, where moderate reaction conditions can be utilized. Such pressures can be in the range of pressures found in typical commercial operations.

Another object of the present invention is to provide a process for preparing a catalyst composition comprising ruthenium where such process does not utilize impregnation techniques and further, such catalyst composition can be contacted with a fluid comprising hydrogen and carbon monoxide to provide high molecular weight hydrocarbons, such as polymethylene, where moderate reaction conditions can be utilized.

Another object of the present invention is to provide a process for preparing a catalyst composition containing less ruthenium than typical supported ruthenium catalyst compositions, yet with less ruthenium, such catalyst composition provides a yield of reaction products such as polymethylene that is equal to or higher compared to catalyst compositions containing more ruthenium prepared by methods other than a process of the present invention.

Another object of the present invention is to provide novel catalyst compositions and processes of producing such catalyst compositions that can be utilized in the production of high molecular weight hydrocarbons, such as polymethylene, from the conversion of fluids comprising hydrogen and carbon monoxide.

An embodiment of the present invention comprises a process of preparing a catalyst composition comprising ruthenium where such process utilizes a novel vapor deposition technique for contacting ruthenium tetraoxide and a support component to provide a catalyst composition that can be contacted, under reaction conditions, with a fluid comprising hydrogen and carbon monoxide.

Another embodiment of the present invention comprises a catalyst composition comprising ruthenium deposited by a novel vapor deposition technique on a support component. The amount of ruthenium deposited is significantly less than the amount of ruthenium utilized when preparing a catalyst composition using impregnation techniques. Novel catalyst compositions of the present invention provide a yield of high molecular weight reaction products such as polymethylene that is equal to or higher compared to catalyst compositions containing more ruthenium prepared by methods other than a process of the present invention.

Other objects and advantages of the present invention will become apparent from the detailed description and the appended claims.

### DETAILED DESCRIPTION OF THE INVENTION

It has been discovered that a novel catalyst composition, comprising ruthenium deposited on a support component according to a process of the present invention, can be contacted with a fluid comprising hydrogen and carbon monoxide to provide for high molecular weight hydrocarbons such as polymethylene that can be utilized under moderate reaction conditions. Further, such catalyst composition provides for higher yields of such high molecular weight hydrocarbons compared to currently used catalyst compositions comprising ruthenium prepared according to processes other than a process of the present invention.

The term “high molecular weight hydrocarbon” as referred to herein includes any hydrocarbon having a molecular weight greater than about 2×10³ molecular weight units (mwu) that can be prepared by a process of the present invention comprising contacting a fluid comprising hydrogen and carbon monoxide with a catalyst composition of the present invention comprising ruthenium and a support component. The term “polymethylene” as referred to herein includes any hydrocarbon materials comprised substantially of methylene (CH₂) fragments catenated or linked in long chains. The long chains may contain branches. The polymethylene material comprises a mixture of various molecular weights.

A catalyst composition of the present invention comprises ruthenium and a support component. In preparing a catalyst composition of the present invention, the ruthenium is initially present as ruthenium tetraoxide that has been deposited under a depositing condition on, in, or with a support component as described herein. The ruthenium tetraoxide is preferably prepared by contacting, under a contacting condition, a strong oxidizing agent and a ruthenium component followed by heating to provide for a gaseous ruthenium tetraoxide that can then be deposited under a depositing condition on a support component. After depositing, the ruthenium tetraoxide-containing support can then be treated under a treating condition as described herein and activated under an activating condition as described herein. A ruthenium component of the present invention, also referred to as a ruthenium tetraoxide precursor, can be any ruthenium component that suitably provides for a catalyst composition of the present invention comprising ruthenium that can be utilized in a process for preparing polymethylene according to a process of the present invention. Examples of a suitable ruthenium component include, but are not limited to, ruthenium bromide, ruthenium bromide hydrate, ruthenium chloride, ruthenium chloride hydrate, ruthenium iodide, ruthenium nitrosyl nitrate, ruthenium oxide, ruthenium oxide hydrate, and the like and combinations thereof. Examples of a preferred ruthenium component that can be utilized in preparing a catalyst composition of the present invention include, but are not limited to, ruthenium chloride, ruthenium chloride hydrate, or ruthenium nitrosyl nitrate. Preferably, a ruthenium component is present in, preferably dissolved in, an aqueous solution such as, but not limited to, deionized water.

A support component of a catalyst composition of the present invention can be any support component that suitably provides for a catalyst composition of the present invention comprising ruthenium that can be utilized in a process of providing polymethylene according to a process of the present invention. A support component of a catalyst composition of the present invention can be any inorganic metal oxide that is typically used as a catalytic support material. Examples of a suitable support component include, but are not limited to, oxides of the metals of Groups II, III, IV, V, and VI A of the Periodic Table of the Elements, and the like and combinations thereof. The oxides of the metals of Groups II, III B and IV B of the Periodic Table of the Elements are preferred, such as alumina, boria, zinc oxide, magnesia, calcium oxide, strontium oxide, barium oxide, titania, zirconia, vanadia, and the like and combinations thereof. More preferred support components include silicon dioxide, zirconyl phosphate, and the like and combinations thereof. The support component can be synthetically prepared or can be a naturally occurring support material, such as naturally occurring clays, kieselguhr, diatomaceous earth, zeolites, silica, thoria, zirconia, and the like and combinations thereof.

An amount of ruthenium component utilized in a process of preparing a catalyst composition of the present invention is such as to provide a concentration of ruthenium in a catalyst composition of the present invention that suitably provides for a catalyst composition of the present invention that can be utilized in preparing polymethylene from a fluid comprising hydrogen and carbon monoxide. An amount of ruthenium component is such as to provide a concentration of ruthenium in a catalyst composition of the present invention generally in the range of from about 0.5 weight percent to about 10 weight percent based on the total weight of the catalyst composition, preferably in the range of from about 0.5 weight percent to about 5 weight percent, and more preferably in the range of from about 1 weight percent to about 5 weight percent.

A support component of a catalyst composition of the present invention can be prepared by any suitable manner or method(s) that suitably provides for a support component of the present invention. Generally, when a support component comprises zirconium present as a zirconyl phosphate, a process of preparing such zirconyl phosphate comprises contacting a zirconyl salt and a water-soluble acidic phosphorous compound, preferably phosphoric acid. Generally, the weight ratio of a zirconyl salt to a water-soluble acidic phosphorous compound, preferably phosphoric acid, is in the range of from about 0.5:1 to about 10:1, preferably in the range of from about 0.5:1 to about 5:1, and more preferably in the range of from about 1:1 to about 5:1.

A water-soluble acidic phosphorous compound utilized in preparing a zirconyl phosphate can be any water-soluble acidic phosphorous compound that suitably provides for a zirconium component, such as zirconyl phosphate, as described herein that can be utilized as a support component in a catalyst composition of the present invention. Examples of a suitable water-soluble acidic phosphorous compound include, but are not limited to, phosphoric acid, phosphorous acid, and the like and combinations thereof. Preferably a water-soluble acidic phosphorous compound is phosphoric acid.

A zirconyl salt suitable for preparing zirconyl phosphate that can be utilized as a support component of a catalyst composition of the present invention can be any zirconyl salt that suitably provides for a catalyst composition of the present invention. Examples of a suitable zirconyl salt include, but are not limited to, zirconyl chloride hydrate, zirconyl chloride octahydrate, zirconyl nitrate hydrate, zirconyl nitrate, zirconyl perchlorate octahydrate, and the like and combinations thereof. Preferably, a zirconyl salt utilized in preparing a zirconyl phosphate that can be utilized as a zirconium component in the preparation of a catalyst composition of the present invention is zirconyl nitrate hydrate.

A process of preparing a zirconyl phosphate that can be utilized as a support component of a catalyst composition of the present invention further comprises contacting a zirconyl salt and a water-soluble acidic phosphorous compound, preferably phosphoric acid, with a hydroxide component that provides for an increase of pH and for precipitating the zirconyl phosphate.

A hydroxide component utilized in preparing a catalyst composition of the present invention can be any hydroxide component that suitably provides for a zirconyl phosphate that can be utilized as a support component in preparing a catalyst composition of the present invention. Examples of a suitable hydroxide component include, but are not limited to, ammonium hydroxide, tetramethyl ammonium hydroxide, tetramethyl ammonium bromide, tetraethyl ammonium bromide, lithium hydroxide, sodium hydroxide, sodium hydrosulfide, potassium hydroxide, calcium hydroxide, magnesium hydroxide, sodium bicarbonate, sodium carbonate, sodium oxide, sodium sulfate, magnesium oxide, calcium oxide, calcium carbonate, sodium phenoxide, barium phenoxide, calcium phenoxide, and the like and combinations thereof. A preferred example of a hydroxide component is ammonium hydroxide.

A weight ratio of hydroxide component to a zirconyl salt and a water-soluble acidic phosphorous compound, preferably phosphoric acid, can be any weight ratio that suitably provides for a zirconyl phosphate that can be utilized as a zirconium component in a catalyst composition of the present invention. Generally the weight ratio of hydroxide component to zirconyl salt and water-soluble acidic phosphorous compound, preferably phosphoric acid, is in the range of from about 0.01:1 to about 1:1, preferably in the range of from about 0.1:1 to about 1:1, and more preferably in the range of from about 0.2:1 to about 0.5:1.

A process of preparing a zirconyl phosphate that can be utilized as a support component in a catalyst composition of the present invention can further comprise contacting, such as washing, the zirconyl phosphate with an aqueous solution and can further comprise contacting, such as washing, with an alcohol. Examples of a suitable aqueous solution include, but are not limited to, deionized water. Examples of a suitable alcohol include, but are not limited to, methyl alcohol, ethyl alcohol, isopropyl alcohol, butyl alcohol, and the like and combinations thereof. A preferred alcohol is methyl alcohol.

A process of preparing a zirconyl phosphate of a catalyst composition of the present invention further comprises drying under a drying condition. A “drying condition” as referred to herein includes a temperature generally in the range of from about 20° C. to about 200° C., preferably in the range of from about 50° C. to about 175° C., and more preferably in the range of from about 50° C. to about 150° C. A drying condition further comprises a pressure generally in the range of from about 0 pounds per square inch absolute (psia) to about 200 psia, preferably in the range of from about 1 psia to about 150 psia, and more preferably in the range of from about 2 psia to about 100 psia. A drying condition further comprises a time period generally in the range of from about 0.5 hour to about 10 hours, preferably in the range of from about 0.5 hour to about 5 hours, and more preferably in the range of from about 1 hour to about 2 hours. A drying condition further comprises an atmosphere, suitable for drying as described herein, preferably air.

A process of preparing a zirconyl phosphate that can be utilized as a support component of a catalyst composition of the present invention further comprises calcining under a calcining condition. A “calcining condition” as referred to herein includes a temperature generally in the range of from about 250° C. to about 1000° C., preferably in the range of from about 300° C. to about 800° C., and more preferably in the range of from about 400° C. to about 600° C. A calcining condition further comprises a pressure generally in the range of from about 0 pounds per square inch absolute (psia) to about 750 psia, preferably in the range of from about 1 psia to about 600 psia, and more preferably in the range of from about 2 psia to about 500 psia. A calcining condition further comprises a time period generally in the range of from about 0.5 hour to about 30 hours, preferably in the range of from about 1 hour to about 20 hours, and more preferably in the range of from about 1 hour to about 10 hours. The calcining can be done in an oxygen-containing atmosphere (e.g., air). During calcining, substantially all volatile matter (e.g., water and carbonaceous materials) is removed.

A process of preparing a zirconyl phosphate that can be utilized as a support component of a catalyst composition of the present invention further comprises contacting the zirconyl phosphate with a silica component. A silica component can be any silica component that suitably provides for a support component of a catalyst composition of the present invention. Examples of a suitable silica component include, but are not limited to, silica, colloidal silica, silica gel, and the like and combinations thereof. Preferably a silica component is silica gel. Preferably, contacting of the zirconyl phosphate with a silica component occurs during contacting of a zirconyl salt and a water-soluble acidic phosphorous compound, preferably phosphoric acid, as described herein. A weight ratio of silica component to zirconyl phosphate can be any weight ratio that suitably provides for a support component of a catalyst composition of the present invention. Generally, a weight ratio of silica component to zirconyl phosphate is generally in the range of from about 0.01:1 to about 1:1, preferably in the range of from about 0.1:1 to about 0.75:1, and more preferably in the range of from about 0.1:1 to about 0.5:1.

When preparing a zirconyl phosphate as described herein, contacting of a zirconyl salt and any water-soluble acidic phosphoric compound, preferably phosphoric acid, can include any contacting technique that suitably provides for a zirconyl phosphate that can be utilized as a support component of a catalyst composition of the present invention. Preferably, such contacting comprises contacting under a precipitating condition that comprises a temperature, pressure, and time period that suitably provides for the precipitation of a zirconium-containing precipitate. A “precipitating condition” as referred to herein includes a temperature generally in the range of from about 20° C. to about 90° C., preferably in the range of from about 20° C. to about 80° C., and more preferably in the range of from about 30° C. to about 70° C. A precipitating condition further includes a pressure generally in the range of from about 0 pounds per square inch absolute (psia) to about 100 psia, preferably in the range of from about 1 psia to about 80 psia, and more preferably in the range of from about 2 psia about 50 psia. A precipitating condition further includes a time period generally in the range of from about 0.1 hour to about 10 hours, preferably in the range of from about 0.1 hour to about 5 hours, and more preferably in the range of from about 0.1 hour to about 2 hours.

For example, when preparing a zirconium-containing precipitate as described herein, a first aqueous solution containing a water-soluble zirconyl salt such as zirconyl nitrate and a water-soluble acidic phosphorous compound, preferably phosphoric acid, is prepared. Any suitable concentrations of these compounds in the aqueous solution can be employed so long as the concentration can result in production of a zirconium-containing precipitate. Generally, a concentration of the compounds in aqueous solution is in the range of from about 0.02 mole/L to about 1 mole/L of each compound. The initial pH of this aqueous solution is generally in the range of from about 1 to about 3. When preparing a zirconyl phosphate that can be utilized as a support component of a catalyst composition of the present invention, an aqueous hydroxide-component containing solution, preferably an aqueous solution of ammonium hydroxide containing in the range of from about 25 weight percent ammonium hydroxide to about 30 weight percent ammonium hydroxide, generally having a pH in the range of from about 10 to about 14, is then added, under a precipitating condition as described herein, to the first aqueous solution containing a water-soluble zirconyl salt and a water-soluble acidic phosphorous compound in an amount sufficient to raise the pH of the first solution to at least 7, preferably in the range of from about 8 to about 9, and to provide for the precipitation of zirconyl phosphate.

The dispersion of the formed zirconium-containing precipitate, preferably zirconyl phosphate, in the aqueous solution is then subjected to any suitable solid-liquid separation method(s) known to one skilled in the art such as, for example, filtration, to substantially separate the precipitate from the aqueous solution to give a separated composition, preferably a separated precipitate comprising zirconyl phosphate. Preferably, the separated precipitate is contacted, such as washed, with water to remove adhered solution therefrom, optionally followed by contacting, such as by washing, with a water-soluble organic solvent such as an alcohol as described herein. The separated precipitate, preferably washed separated precipitate, is generally dried under a drying condition as described herein to provide a dried composition, preferably a dried precipitate, and can be calcined under a calcining condition as described herein.

A process of preparing a catalyst composition of the present invention generally comprises contacting a ruthenium component as described herein with an oxidizing agent that provides for a gaseous ruthenium tetraoxide that can be deposited under a depositing condition on, in, or with a support component as described herein. An oxidizing agent can be any oxidizing agent that provides for the production of a gaseous ruthenium tetraoxide when such oxidizing agent is contacted with a ruthenium component according to a process of the present invention. Examples of suitable oxidizing agents include, but are not limited to, potassium meta-periodate, periodic acid, potassium permanganate, perchloric acid, potassium chromate, chromic acid, and the like and combinations thereof. A preferred oxidizing agent is potassium meta-periodate.

Contacting of a ruthenium component and an oxidizing agent comprises any contacting manner or method(s) that suitably provides for the production of gaseous ruthenium tetraoxide. Preferably, contacting comprises mixing. Generally, the weight ratio of a ruthenium component to an oxidizing agent is in the range of from about 0.5:1 to about 10:1, preferably in the range of from about 0.5:1 to about 5:1, and more preferably in the range of from about 1:1 to about 5:1. The contacting of a ruthenium component and an oxidizing agent as described herein can be conducted in the presence of an aqueous solution such as, but not limited to, deionized water. When a ruthenium component of the present invention is present in an aqueous solution before contacting with an oxidizing agent as described herein, the concentration of the ruthenium component in the aqueous solution is in the range of from about 0.1 gm/mL to about 1 gm/mL, preferably in the range of from about 0.2 gm/mL to about 0.5 gm/mL. The presently preferred ruthenium component to be used in the aqueous solution to be contacted with an oxidizing agent as described herein is ruthenium chloride hydrate.

Generally, if the contacting of a ruthenium component and an oxidizing agent as described herein is vigorous enough, the general exothermic nature of the reaction of the ruthenium component and oxidizing agent will provide for the production, also referred to as release, of gaseous ruthenium tetraoxide. Preferably, to aid in the production or release of ruthenium tetraoxide during contacting of a ruthenium component and an oxidizing agent as described herein, the mixture of ruthenium component and an oxidizing agent are subjected to heating under a heating condition. A “heating condition” as referred to herein includes a temperature generally in the range of from about 20° C. to about 200° C., preferably in the range of from about 20° C. to about 150° C., and more preferably in the range of from about 30° C. to about 100° C. A heating condition further includes a pressure generally in the range of from about 0 pounds per square inch absolute (psia) to about 50 psia, preferably in the range of from about 1 psia to about 40 psia, and more preferably in the range of from about 2 psia to about 30 psia. A heating condition further includes a time period generally in the range of from about 0.1 hour to about 10 hours, preferably in the range of from about 0.1 hour to about 5 hours, and more preferably in the range of from about 0.1 hour to about 2 hours. Heating can be provided by any manner or method(s) known in the art for heating a mixture such as, but not limited to, thermostatically-controlled resistance heating, and the like and combinations thereof. Gaseous ruthenium tetraoxide is released from the heated mixture of ruthenium component and oxidizing agent.

Gaseous ruthenium tetraoxide is then deposited under a depositing condition on, in, or with a support component of the present invention. A depositing condition can be any condition that suitably provides for the depositing of gaseous ruthenium tetraoxide on, in, or with a support component of the present invention. A “depositing condition” as referred to herein includes a temperature generally in the range of from about 20° C. to about 200° C., preferably in the range of from about 20° C. to about 150° C., and more preferably in the range of from about 30° C. to about 100° C. A depositing condition further includes a pressure generally in the range of from about 0 pounds per square inch absolute (psia) to about 50 psia, preferably in the range of from about 1 psia to about 40 psia, and more preferably in the range of from about 2 psia to about 30 psia. A depositing condition further includes a time period generally in the range of from about 0.1 hour to about 10 hours, preferably in the range of from about 0.1 hour to about 5 hours, and more preferably in the range of from about 0.1 hour to about 2 hours. Preferably, depositing under a depositing condition comprises the use of a carrier gas that is passed through or over the mixture of a ruthenium component and an oxidizing agent as described herein. A carrier gas of a process of the present invention can be any carrier gas suitable for providing a depositing of gaseous ruthenium tetraoxide on, in, or with a support component of the present invention. Examples of a suitable carrier gas include, but are not limited to, nitrogen, argon, and the like and combinations thereof. A preferred carrier gas is nitrogen. The carrier gas can also be used to help maintain the pressure of the depositing condition as described herein.

An apparatus for depositing ruthenium tetraoxide on, in, or with a support component as described herein can be any apparatus known in the art capable of depositing a gaseous substance or compound on, in, or with a support component. Examples of suitable depositing apparatus include, but are not limited to, hot wall reactors, cold wall reactors, radiation beam assisted reactors, plasma assisted reactors, and the like and combinations thereof.

After contacting, preferably depositing, ruthenium tetraoxide on, in, or with a support component as described herein, the resulting catalyst composition of the present invention can be subjected to treating under a treating condition. Also, if desired, after contacting, preferably depositing, ruthenium tetraoxide on, in, or with a support component as described herein, the resulting catalyst composition of the present invention can be dried under a drying condition as described herein before treating under a treating condition. A “treating condition” as referred to herein includes a temperature generally in the range of from about 100° C. to about 500° C., preferably in the range of from about 150° C. to about 400° C., and more preferably in the range of from about 150° C. to about 300° C. A treating condition further comprises a pressure generally in the range of from about 0 pounds per square inch absolute (psia) to about 100 psia, preferably in the range of from about 1 psia to about 50 psia, and more preferably in the range of from about 2 psia to about 30 psia. A treating condition further comprises a time period generally in the range of from about 0.1 hour to about 20 hours, preferably in the range of from about 0.1 hour to about 10 hours, and more preferably in the range of from about 0.5 hour to about 5 hours.

A treating condition further comprises an atmosphere selected from the group consisting of an oxygen-containing atmosphere (e.g., air), nitrogen, argon, and the like and combinations thereof. Generally, the treating can immediately follow the depositing under a depositing condition as described herein by allowing the carrier gas to continue to flow under a treating condition after the release of ruthenium tetraoxide is substantially complete.

A process of preparing a catalyst composition of the present invention further comprises, after contacting, preferably depositing, ruthenium tetraoxide on, in, or with a support component as described herein and preferably after treating under a treating condition as described herein, activating under an activating condition that suitably provides for a catalyst composition that can be utilized in a process of the present invention for producing polymethylene from a fluid comprising hydrogen and carbon monoxide. It should be understood that the ruthenium tetraoxide may be converted to ruthenium during the activating under an activating condition as described herein. An “activating condition” as referred to herein includes a temperature generally in the range of from about 50° C. to about 500° C., preferably in the range of from about 60° C. to about 400° C., and more preferably in the range of from about 70° C. to about 300° C. An activating condition further comprises a pressure generally in the range of from about 0 pounds per square inch absolute (psia) to about 750 psia, preferably in the range of from about 1 psia to about 500 psia, and more preferably in the range of from about 2 psia to about 400 psia. An activating condition as referred to herein further comprises a time period generally in the range of from about 0.1 hour to about 30 hours, preferably in the range of from about 0.5 hour to about 20 hours, and more preferably in the range of from about 1 hour to about 10 hours. An activating condition further comprises an atmosphere suitable for activating a catalyst composition of the present invention. Examples of a suitable activating atmosphere include, but are not limited to, hydrogen, carbon monoxide, synthesis gas, other reducing gases, and the like and combinations thereof. A preferred activating atmosphere is hydrogen. If desired, treating under a treating condition as described herein and activating under an activating condition as described herein can occur simultaneously.

A catalyst composition of the present invention can have any suitable shape or form. Preferably, a catalyst composition of the present invention is in the form of tablets, pellets, extrudates, spheres, and the like and combinations thereof. A catalyst composition of the present invention generally has a particle size in the range of from about 50 micrometers to about 10 millimeters (mm), preferably in the range of from about 55 micrometers to about 8 mm, and more preferably in the range of from about 60 micrometers to about 6 mm.

A catalyst composition of the present invention can be used in a process of the present invention comprising contacting, under reaction conditions, a catalyst composition with a fluid comprising hydrogen and carbon monoxide. The term “fluid” as referred to herein denotes gas, liquid, vapor, and combinations thereof.

Generally, a mole ratio of hydrogen to carbon monoxide can be any mole ratio that provides for a fluid that can be contacted with a catalyst composition of the present invention to provide high molecular weight hydrocarbons, preferably polymethylene. Generally, the mole ratio of hydrogen to carbon monoxide is in the range of from about 1:1 to about 5:1, preferably in the range of from about 1:1 to about 4:1, more preferably in the range of from about 1:1 to about 3:1, and most preferably the mole ratio is about 2:1.

Reaction conditions of a process of the present invention can be any reaction conditions that suitably provide for the production of polymethylene from a fluid comprising hydrogen and carbon monoxide according to a process of the present invention. The reaction conditions generally comprise a temperature generally in the range of from about 100° C. to about 500° C., preferably in the range of from about 100° C. to about 400° C., and more preferably in the range of from about 100° C. to about 300° C. The reaction conditions further comprise a pressure generally in the range of from about 500 pounds per square inch gauge (psig) to about 10,000 psig, preferably in the range of from about 750 psig to about 7500 psig, and more preferably in the range of from about 1000 psig to about 5000 psig. The reaction conditions further comprise a charge rate of fluid such that the weight hourly space velocity is generally in the range of from about 0.01 hour⁻¹ to about 1000 hour⁻¹, preferably in the range of from about 0.05 hour⁻¹ to about 750 hour⁻¹, and more preferably in the range of from about 0.1 hour⁻¹ to about 500 hour⁻¹.

A process of the present invention can further comprise contacting the catalyst composition and fluid comprising hydrogen and carbon monoxide in the presence of a solvent. Examples of a suitable solvent include, but are not limited to, cyclopentane, cyclohexane, cycloheptane, cyclooctane, and the like and combinations thereof, preferably cyclohexane. Generally, a weight ratio of solvent to catalyst composition can be any weight ratio that provides for the preparation of polymethylene from a fluid comprising hydrogen and carbon monoxide. A weight ratio of solvent, preferably cyclohexane, to catalyst composition is generally in the range of from about 400:1 to about 20:1, preferably in the range of from about 300:1 to about 50:1, and more preferably in the range of from about 200:1 to about 80:1.

The fluid comprising hydrogen and carbon monoxide can be contacted by any suitable means, method(s), or manner with a catalyst composition of the present invention as described herein contained within a reaction zone. The contacting step can be operated as a batch process step or, preferably, as a continuous process step, preferably in a slurry phase reactor. In the latter operation, a solid catalyst bed, a moving catalyst bed, a fluidized catalyst bed, or a bubble slurry bed can be employed. Any of these operational modes have advantages and disadvantages, and those skilled in the art can select the one most suitable for a particular fluid and catalyst composition. The contacting step is preferably carried out within a reaction zone comprising a slurry phase reactor wherein is contained a catalyst composition of the present invention and under reaction conditions that suitably promote the production of high molecular weight hydrocarbons comprising polymethylene from at least a portion of the fluid.

Generally, the process effluent from the reaction zone, also referred to as a contacting zone, can be separated into the principal fractions such as high molecular weight hydrocarbons comprising polymethylene and lighter fractions such as alcohols and lower molecular weight hydrocarbons, by any known method(s) such as, for example, fractionation distillation. Because the separation methods are well-known to one skilled in the art, the description of such separation methods is omitted herein.

After a catalyst composition of the present invention has been deactivated by, for example, coke deposition or feed poisons, to the extent that the fluid conversion and/or the selectivity to polymethylene has become unsatisfactory, the catalyst composition can be reactivated by any means or method(s) known to one skilled in the art such as, for example, calcining in air to bum off deposited coke and other carbonaceous materials, such as oligomers or polymers, preferably at a temperature in the range of from about 400° C. to about 1000° C. The optimal time periods of calcining depend generally on the types and amounts of deactivating deposits on the catalyst composition and on the calcination temperatures. These optimal time periods can easily be determined by those possessing ordinary skill in the art and are omitted herein for the interest of brevity.

The following examples are presented to further illustrate the present invention and are not to be construed as unduly limiting the scope of the present invention.

### EXAMPLE I

This example illustrates the preparation of several catalyst compositions that were subsequently tested as catalysts in the preparation of polymethylene.

CATALYST A: A 5.82 gram quantity of ruthenium chloride hydrate (RuCl₃.2H₂O) (obtained from Aldrich Chemical Company, Milwaukee, Wis. with a product designation 46,377-9) was dissolved in about 50 mL of deionized water using an ultrasonic probe for dispersion and to form a black slurry. The slurry was then added incrementally to a 15 gram quantity of silica gel (obtained from Aldrich Chemical Company, Milwaukee, Wis. with a product designation of 28,862-4 with a less than 60 mesh, 250 micron size) using an incipient wetness impregnation technique, i.e., essentially completely filling the pores of the silica gel. Drying was conducted at atmospheric pressure (about 14.7 psia) and room temperature (about 25° C.) between additions and, after the final addition, drying was conducted in a vacuum oven at 120° C. for about 1 hour. A 3.04 gram quantity of the resulting dried composition was then placed on a frit contained within a calcining tube located in a furnace. Gas flowed to the calcining tube through a one-eighth inch stainless steel induction tube and up through the frit. Depending on the desired contactings and treatings to be conducted, the gas type and flow could be varied. Ammonium hydroxide could also be added using a metering pump. After contact with the material(s) on the frit, the various gas flows exited the furnace and into a 500 mL Drechsel gas washing bottle and then vented to atmosphere. The conduits to and from the furnace were one-eighth inch TEFLON tubing. The 3.04 gram quantity of the resulting dried composition was treated at about 250° C. in a nitrogen atmosphere for about 20 minutes. The gas flow was then changed to hydrogen and the process was continued at about 250° C. for about 4 hours and 20 minutes to reduce the ruthenium. Catalyst A contained about 13.8 weight percent ruthenium and about 14 weight percent chloride based on analysis.

CATALYST B: A 5.17 gram quantity of ruthenium chloride hydrate (RuCl₃.2H₂O) (obtained from Aldrich Chemical Company, Milwaukee, Wis. with a product designation 28,862-4) was dissolved in 15 mL of deionized water using an ultrasonic probe for dispersion and to form a slurry. The slurry was then added incrementally to a 12.09 gram quantity of silica gel (obtained from Aldrich Chemical Company, 70-230 mesh) with stirring. After the final addition, the resulting composition was allowed to dry in a vacuum oven at about 50° C. for about 16 hours. The resulting dried composition was then sieved to pass a 60 mesh screen to provide a sieved composition denoted herein as “Sieved Composition”. A 2.5 gram quantity of the Sieved Composition was then placed on a frit contained within a calcining tube located in a furnace as described herein for Catalyst A. The Sieved Composition was treated at about 250° C. in a nitrogen atmosphere for about 1 hour. The gas was switched to hydrogen and the process was continued for about 1 hour. The resulting composition product was then treated for about 1 hour with 10 mL of 0.066 M ammonium hydroxide (NH₄OH) while being maintained at about 250° C. Hydrogen flow was then reintroduced for about 1 hour. Hydrogen flow was then stopped and nitrogen was used to cool the resulting composition. Catalyst B contained about 18.1 weight percent ruthenium and about 0.78 weight percent chloride based on analysis.

CATALYST C: A 1.96 gram quantity of non-ammonium hydroxide treated Catalyst B described herein was placed on a frit contained within a calcining tube located in a furnace as described herein for Catalyst A and treated at about 250° C. in a nitrogen atmosphere for about 5 minutes. The gas was then switched to hydrogen and the process was continued at about 250° C. for about 3 hours to reduce the ruthenium. Catalyst C contained about 14.9 weight percent ruthenium and about 13 weight percent chloride based on analysis.

CATALYST D: A 40.32 gram quantity of zirconyl nitrate hydrate (Zr(O)(NO₃)₂.2H₂O) (obtained from Aldrich Chemical Company, Milwaukee, Wis. with a product designation 38,067-9) was placed in a 500 mL beaker with 300 mL deionized water and heated on a stirring hot plate with a magnetic stirrer to dissolve the zirconyl nitrate hydrate. Stirring was continued and a 20 gram quantity of silicon dioxide (obtained from Aldrich Chemical Company, 70-230 mesh) was added. A 13.4 mL quantity of phosphoric acid (85 weight percent H₃PO₄ in water obtained from Aldrich Chemical Company, Milwaukee, Wis. with a product designation 21,510-4) was then added using a dropping funnel. A sol-gel precipitate formed below a pH of 2. With heating and stirring, ammonium hydroxide (NH₄OH) (obtained from Aldrich Chemical Company, Milwaukee, Wis. with a product designation 33,881-8) was added until the pH was above 8. Stirring was continued for an additional 30 minutes. The resulting filtered precipitate was washed by stirring with a 300 mL quantity of deionized water, filtered again, washed again with isopropyl alcohol, then filtered and dried in a vacuum oven at about 200° C. for about 16 hours. The resulting dried precipitate was then ground to pass a 60 mesh screen. A 5.0 gram quantity of the ground material comprising zirconyl phosphate and silicon dioxide was then placed on a frit contained within a calcining tube located in a furnace in preparation for depositing a gaseous ruthenium tetraoxide on the zirconyl phosphate and silicon dioxide support. Gas flow was transported to the calcining tube through a one-eighth inch stainless steel induction tube and down through the frit. Depending on the desired contactings and treatings to be conducted, the gas type and flow could be varied. After contact with the material(s) on the frit, the various gas flows exited the furnace and into a 500 mL Drechsel gas washing bottle and then vented to atmosphere. The conduits to and from the furnace were one-eighth inch TEFLON tubing.

A 5.39 gram quantity of ruthenium chloride hydrate (RuCl₃.2H₂O) (obtained from Aldrich Chemical Company, Milwaukee, Wis. with a product designation 28,862-4) and 100 mL of deionized water were then added to a 500 mL Drechsel gas washing bottle equipped with a magnetic stirrer and wrapped with three-eighth inch copper tubing. The copper tubing was connected to a Thermo NESLAB RTE-11 heating/cooling unit (Thermo NESLAB, Portsmouth, N.H.). The gas washing bottle and copper tubing were positioned in a 3000 mL beaker that was half filled with deionized water. The gas washing bottle, copper tubing, and beaker were set on a stirrer/hot plate as a unit. A 15.51 gram quantity of potassium meta-periodate (KIO₄) (obtained from Aldrich Chemical Company, Milwaukee, Wis. with a product designation 21,005-6) and 250 mL of deionized water were then added to an addition funnel, equipped with pressure equalizing lines, that was connected to the gas washing bottle located in the beaker. The temperature of the ruthenium chloride hydrate dispersion was increased to 55° C. using the hot plate and the temperature was maintained with the Thermo NESLAB RTE-11 heating/cooling unit. Stirring was maintained with the magnetic stirrer.

Nitrogen gas was then passed through the addition funnel containing the potassium meta-periodate dispersion to provide for the transporting of potassium meta-periodate to the ruthenium chloride hydrate dispersion to provide for a contacting of the potassium meta-periodate and ruthenium chloride hydrate and for the release of gaseous ruthenium to tetraoxide. The gaseous ruthenium tetraoxide was then transported by the nitrogen gas to the calcining tube that contained the 5.0 gram quantity of the ground material comprising zirconyl phosphate and silicon dioxide. The furnace temperature was about 125° C. Periodically, the flow of nitrogen and ruthenium tetraoxide was stopped: and hydrogen was admitted to the support to reduce the ruthenium tetraoxide. The addition of the solution in the gas washing bottle located in the beaker was substantially complete after about 3 hours and 15 minutes. The nitrogen and hydrogen flows were continued for about 1 hour. Catalyst D contained about 4.09 weight percent ruthenium based on analysis.

CATALYST E: Catalyst E was prepared in a manner similar to Catalyst D, except the support comprised silicon dioxide and did not contain zirconyl phosphate. A 5.0 gram quantity of silicon dioxide (obtained from Aldrich Chemical Company, Milwaukee, Wis., 70-230 mesh) was placed on a frit contained within a calcining tube located in a furnace as described herein for Catalyst D in preparation for depositing gaseous ruthenium tetraoxide on the silicon dioxide support. A 1.01 gram quantity of ruthenium chloride hydrate (RuCl₃.2H₂O) (obtained from Aldrich Chemical Company, Milwaukee, Wis. with a product designation 28,862-4) and 100 mL of deionized water were then added to a 500 mL Drechsel gas washing bottle equipped with a magnetic stirrer and wrapped with three-eighth inch copper tubing. The copper tubing was connected to a Thermo NESLAB RTE-11 heating/cooling unit (Thermo NESLAB, Portsmouth, N.H.). The gas washing bottle and copper tubing were positioned in a 3000 mL beaker that was half filled with deionized water. The gas washing bottle, copper tubing, and beaker were set on a stirrer/hot plate as a unit. A 3.74 gram quantity of potassium meta-periodate (KIO₄) (obtained from Aldrich Chemical Company, Milwaukee, Wis. with a product designation 21,005-6) and 100 mL of deionized water were then added to an addition funnel, equipped with pressure equalizing lines, that was connected to the gas washing bottle located in the beaker. The temperature of the ruthenium chloride hydrate dispersion was increased to 55° C. using the hot plate and the temperature was maintained with the Thermo NESLAB RTE-11 heating/cooling unit. Stirring was maintained with the magnetic stirrer.

Nitrogen gas was then passed through the addition funnel containing the potassium meta-periodate dispersion to provide for the transporting of potassium meta-periodate to the ruthenium chloride hydrate dispersion to provide for a contacting of the potassium meta-periodate and ruthenium chloride hydrate and for the release of gaseous ruthenium tetraoxide. The gaseous ruthenium tetraoxide was then transported by the nitrogen gas to the calcining tube that contained the 5.0 gram quantity of silicon dioxide. The furnace temperature was about 125° C. Periodically, the flow of nitrogen and ruthenium tetraoxide was stopped and hydrogen was admitted to the support to reduce the ruthenium tetraoxide. The addition of the solution in the gas washing bottle located in the beaker was substantially complete after about 3 hours and 15 minutes. The nitrogen and hydrogen flows were continued for about 1 hour. Catalyst E contained about 1.3 weight percent ruthenium based on analysis.

### EXAMPLE II

This example illustrates the use of the catalyst compositions described herein in Example I as catalyst compositions in the preparation of high molecular weight hydrocarbons, such as polymethylene, from a fluid comprising hydrogen and carbon monoxide.

An approximately 2.0 gram sample of each of the catalyst compositions described in Example I was reduced at about 250° C. for about two hours with hydrogen. For each test, approximately 1.85 grams of each reduced sample were placed into a 300 cc stainless steel autoclave (Autoclave Engineers, Inc., Erie, Pa., Model BC00305505AH, pressure range: less than 6000 psig at 650° F.) with about 180 mL of cyclohexane. The reactor was closed and purged with nitrogen by pressurizing to 300 psig and then depressurizing to 0 psig over a time period of about 5 minutes. The nitrogen pressurizing and depressurizing was repeated four times. The reactor was then purged in a similar manner three times with a fluid containing hydrogen and carbon monoxide in a 2:1 mole ratio. The reactor was then purged in a similar manner three times with a fluid containing hydrogen and carbon monoxide in a 2:1 mole ratio while the mixer was turned on at 500 revolutions per minute for about 15 minutes following each purge. After purging, the reactor was pressurized to 1100 psig with a fluid containing hydrogen and carbon monoxide in a 2:1 mole ratio and heated to about 150° C. with the mixer at 500 revolutions per minute. The reactor pressure was maintained at about 1500 psig by adding the fluid containing hydrogen and carbon monoxide in a 2:1 mole ratio. Fluid uptake was monitored periodically during the run by stopping flow from the fluid feed tank and observing the change in pressure at the reactor. After several hours, the reaction was stopped. The cyclohexane and catalyst composition were separated from the polymer. The polymer was analyzed by gel permeation chromatography. Results of the tests for catalyst compositions A through E are summarized herein in Tables I and II. Table II discloses various characteristics of the product obtained utilizing the catalyst compositions. The productivity of the catalyst composition is reported as gm polymer/gm Ru/hr, which is grams of polymer produced per grams of ruthenium present per hour of reaction. Also noted were the synthesis gas (H₂+CO) uptakes (8 and 12 hour averages) (dPsi/min denotes differential in pressure/minute), the average molecular weight of the product (Mw), the average molecular number of the product (Mn), and the melting point range of the product.

TABLE I

| | | | | | Syn gas | Syn gas |
|---|---|---|---|---|---|---|
| | | | | Reactor | uptake | uptake |
| | | | Ruthenium | temp | dPsi/min | dPsi/min |
| Catalyst | Catalyst/Support | Cat. Prep Method | (wt. %) | (° C.) | (8 hr avg) | (12 hr avg) |
| A | Ru/SiO₂ | RuCl₃ | 13.8 | 156 | 3.4 | 3.4 |
| B | Ru/SiO₂ | RuCl₃ w/NH₃ strip | 18.1 | 148 | 5.4 | 4.3 |
| C | Ru/SiO₂ | RuCl₃ | 14.9 | 144 | 7.0 | 4.7 |
| D | Ru/(Zr(O))₃(PO₄)₂/SiO₂ | RuO₄ | 4.09 | 156 | 2.5 | 2.4 |
| E | Ru/SiO₂ | RuO₄ | 1.3 | 155 | 1.0 | 1.9 |

TABLE II

| | Catalyst | | | | |
|---|---|---|---|---|---|
| | Productivity | | | | |
| | (gm polymer/ | Melting | | | |
| Catalyst | gm Ru/hr) | Point (° C.) | Mw/1000 | Mn/1000 | Mw/Mn |
| A | 0.153 | 112-118 | 4.2 | 1.8 | 2.4 |
| B | 0.300 | 116-118 | — | — | — |
| C | 0.583 | 108-110 | 3.2 | 1.3 | 2.5 |
| D | 0.150 | 116-120 | 5.6 | 2.5 | 2.3 |
| E | 0.830 | 113-120 | 5.1 | 2.6 | 2.0 |

The test data presented herein in Tables I and II clearly show that catalyst compositions comprising ruthenium prepared according to a process of the present invention provide higher yields of polymethylene than catalyst compositions comprising more ruthenium that are prepared by processes other than the inventive process(es) described herein. Further, the data clearly show that the specific sequence of preparing the catalyst composition affects the amount of polymethylene produced.

The results shown in the above examples clearly demonstrate that the present invention is well adapted to carry out the objects and obtain the ends and advantages mentioned as well as those inherent therein.

Reasonable variations, modifications, and adaptations can be made within the scope of the disclosure and the appended claims without departing from the scope of the present invention.

What is claimed is:

1. A process of preparing a high molecular weight hydrocarbon comprising contacting, under reaction conditions, a catalyst composition and a fluid comprising hydrogen and carbon monoxide wherein said catalyst composition is prepared by a process of preparing said catalyst composition comprising contacting a ruthenium component and an oxidizing agent to provide a ruthenium tetraoxide.

2. A process according to claim 1 further comprising depositing under a depositing condition said ruthenium tetraoxide on, in, or with a support component.

3. A process according to claim 1 wherein said high molecular weight hydrocarbon comprises a molecular weight greater than about 2×10³ molecular weight units.

4. A process according to claim 1 wherein said high molecular weight hydrocarbon comprises polymethylene.

5. A process according to claim 1 wherein a mole ratio of said hydrogen to said carbon monoxide is in the range of from about 1:1 to about 5:1.

6. A process according to claim 1 wherein said reaction conditions comprise:
a temperature in the range of from about 100° C. to about 500° C.,
a pressure in the range of from about 500 pounds per square inch gauge to about 10,000 pounds per square inch gauge, and
a charge rate of said fluid such that the weight hourly space velocity is in the range of from about 0.01 hour⁻¹ to about 1000 hour⁻¹.

7. A process according to claim 1 wherein said contacting of said catalyst composition and said fluid is conducted in the presence of a solvent selected from the group consisting of cyclopentane, cyclohexane, cycloheptane, cyclooctane, and combinations thereof.

8. A process according to claim 7 wherein a weight ratio of said solvent to catalyst composition is in the range of from about 400:1 to about 20:1.

9. A process according to claim 1 wherein said process of preparing a high molecular weight hydrocarbon is conducted in a slurry phase reactor.

10. A process according to claim 1 wherein said ruthenium component is selected from the group consisting of ruthenium bromide, ruthenium bromide hydrate, ruthenium chloride, ruthenium chloride hydrate, ruthenium iodide, ruthenium nitrosyl nitrate, ruthenium oxide, ruthenium oxide hydrate, and combinations thereof.

11. A process according to claim 1 wherein said ruthenium component comprises ruthenium chloride, ruthenium chloride hydrate, or ruthenium nitrosyl nitrate.

12. A process according to claim 1 wherein said oxidizing agent is selected from the group consisting of potassium meta-periodate, periodic acid, potassium permanganate, perchloric acid, potassium chromate, chromic acid, and combinations thereof.

13. A process according to claim 12 wherein said oxidizing agent is potassium meta-periodate.

14. A process according to claim 1 wherein a weight ratio of said ruthenium component to said oxidizing agent is in a range of from about 0.5:1 to about 10:1.

15. A process according to claim 1 wherein an amount of said ruthenium component is such as to provide a concentration of ruthenium in said catalyst composition in the range of from about 0.5 weight percent to about 10 weight percent based on the total weight of said catalyst composition.

16. A process according to claim 1 wherein said process of preparing said catalyst composition further comprises heating under a heating condition comprising:
a temperature in the range of from about 20° C. to about 200° C.,
a pressure in the range of from about 0 pounds per square inch absolute to about 50 pounds per square inch absolute, and
a time period in the range of from about 0.1 hour to about 10 hours.

17. A process according to claim 2 wherein said depositing condition comprises:
a temperature in the range of from about 20° C. to about 200° C.,
a pressure in the range of from about 0 pounds per square inch absolute to about 50 pounds per square inch absolute, and
a time period in the range of from about 0.1 hour to about 10 hours.

18. A process according to claim 17 wherein said depositing condition further comprises the use of a carrier gas selected from the group consisting of nitrogen, argon, and combinations thereof.

19. A process according to claim 1 wherein said process of preparing said catalyst composition can be conducted in an apparatus selected from the group consisting of hot wall reactors, cold wall reactors, radiation beam assisted reactors, plasma assisted reactors, and combinations thereof.

20. A process according to claim 2 wherein said support component is selected from the group consisting of oxides of the metals of Groups II, III, IV, V, and VI A of the Periodic Table of the Elements, and combinations thereof.

21. A process according to claim 20 wherein said support component is selected from the group consisting of the oxides of the metals of Groups II, III B and IV B of the Periodic Table of the Elements, and combinations thereof.

22. A process according to claim 21 wherein said support component is selected from the group consisting of alumina, boria, zinc oxide, magnesia, calcium oxide, strontium oxide, barium oxide, titania, zirconia, vanadia, and combinations thereof.

23. A process according to claim 22 wherein said support component is selected from the group consisting of silicon dioxide, zirconyl phosphate, and combinations thereof.

24. A process according to claim 2 further comprises treating under a treating condition comprising:
a temperature in the range of from about 100° C. to about 500° C.,
a pressure in the range of from about 0 pounds per square inch absolute to about 100 pounds per square inch absolute,
a time period in the range of from about 0.1 hour to about 20 hours, and
further wherein said treating condition comprises an atmosphere selected from the group consisting of an oxygen-containing atmosphere, nitrogen, argon, and combinations thereof.

25. A process according to claim 2 further comprises activating under an activating condition comprising:
a temperature in the range of from about 50° C. to about 500° C.,
a pressure in the range of from about 0 pounds per square inch absolute to about 750 pounds per square inch absolute,
a time period in the range of from about 0.1 hour to about 30 hours, and
further wherein said activating condition comprises an atmosphere selected from the group consisting of hydrogen, carbon monoxide, synthesis gas, other reducing gases, and combinations thereof.

26. A process according to claim 23 wherein said zirconyl phosphate is prepared by a process of preparing comprising contacting a zirconyl salt and a water-soluble acidic phosphorous compound.

27. A process according to claim 26 wherein said water-soluble acidic phosphorous compound is selected from the group consisting of phosphoric acid, phosphorous acid, and combinations thereof.

28. A process according to claim 27 wherein said water-soluble acidic phosphorous compound is phosphoric acid.

29. A process according to claim 26 wherein a weight ratio of said zirconyl salt to said water-soluble acidic phosphorous compound is in the range of from about 0.5:1 to about 10:1.

30. A process according to claim 26 wherein said process of preparing said zirconyl phosphate further comprises contacting said zirconyl salt and water-soluble acidic phosphorous compound with a hydroxide component under a precipitating condition to provide for an increase of pH and for precipitating said zirconyl phosphate.

31. A process according to claim 30 wherein said precipitating condition comprises:
a temperature in the range of from about 20° C. to about 90° C.,
a pressure in the range of from about 0 pounds per square inch absolute to about 100 pounds per square inch absolute, and
a time period in the range of from about 0.1 hour to about 10 hours.

32. A process according to claim 26 wherein said zirconyl salt is selected from the group consisting of zirconyl chloride hydrate, zirconyl chloride octahydrate, zirconyl nitrate hydrate, zirconyl nitrate, zirconyl perchlorate octahydrate, and combinations thereof.

33. A process according to claim 26 wherein said zirconyl salt comprises zirconyl nitrate hydrate.

34. A process according to claim 30 wherein said hydroxide component is selected from the group consisting of ammonium hydroxide, tetramethyl ammonium hydroxide, tetramethyl ammonium bromide, tetraethyl ammonium bromide, lithium hydroxide, sodium hydroxide, sodium hydrosulfide, potassium hydroxide, calcium hydroxide, magnesium hydroxide, sodium bicarbonate, sodium carbonate, sodium oxide, sodium sulfate, magnesium oxide, calcium oxide, calcium carbonate, sodium phenoxide, barium phenoxide, calcium phenoxide, and combinations thereof.

35. A process according to claim 30 wherein said hydroxide component comprises ammonium hydroxide.

36. A process according to claim 30 wherein a weight ratio of said hydroxide component to said zirconyl salt and water-soluble acidic phosphorous compound is in the range of from about 0.01:1 to about 1:1.

37. A process according to claim 30 wherein said process of preparing said zirconyl phosphate further comprises contacting said zirconyl phosphate with an aqueous solution.

38. A process according to claim 30 wherein said process of preparing said zirconyl phosphate further comprises contacting with an alcohol selected from the group consisting of methyl alcohol, ethyl alcohol, isopropyl alcohol, butyl alcohol, and combinations thereof.

39. A process according to claim 30 wherein said process of preparing said zirconyl phosphate further comprises drying under a drying condition comprising:
a temperature in the range of from about 20° C. to about 200° C.,
a pressure in the range of from about 0 pounds per square inch absolute to about 200 pounds per square inch absolute,
a time period in the range of from about 0.5 hour to about 10 hours, and
further wherein said drying condition comprises an atmosphere comprising air.

40. A process according to claim 30 wherein said process of preparing said zirconyl phosphate further comprises calcining under a calcining condition comprising:
a temperature in the range of from about 250° C. to about 1000° C.,
a pressure in the range of from about 0 pounds per square inch absolute to about 750 pounds per square inch absolute,
a time period in the range of from about 0.5 hour to about 30 hours, and
further wherein said calcining condition comprises an atmosphere comprising air.

41. A process according to claim 30 wherein said process of preparing said zirconyl phosphate further comprises contacting said zirconyl phosphate with a silica component selected from the group consisting of silica, colloidal silica, silica gel, and combinations thereof.

42. A process according to claim 41 wherein said silica component is silica gel.

43. A process according to claim 41 wherein said contacting said zirconyl phosphate with a silica component occurs during said precipitating of said zirconyl phosphate.

44. A process according to claim 41 wherein a weight ratio of said silica component to said zirconyl phosphate is in the range of from about 0.01:1 to about 1:1.

45. A process of preparing a high molecular weight hydrocarbon comprising contacting, under reaction conditions, a catalyst composition comprising ruthenium and a support component and a fluid comprising hydrogen and carbon monoxide wherein said ruthenium is initially present as ruthenium tetraoxide provided by contacting a ruthenium component and an oxidizing agent to provide said ruthenium tetraoxide that is deposited under a depositing condition on, in, or with said support component.

46. A process according to claim 45 wherein said high molecular weight hydrocarbon comprises a molecular weight greater than about 2×10³ molecular weight units.

47. A process according to claim 45 wherein said high molecular weight hydrocarbon comprises polymethylene.

48. A process according to claim 45 wherein a concentration of said ruthenium in said catalyst composition is in a range of from about 0.5 weight percent to about 10 weight percent based on the total weight of said catalyst composition.

49. A process according to claim 45 wherein a mole ratio of said hydrogen to said carbon monoxide is in the range of from about 1:1 to about 5:1.

50. A process according to claim 45 wherein said reaction conditions comprise:
a temperature in the range of from about 100° C. to about 500° C.,
a pressure in the range of from about 500 pounds per square inch gauge to about 10,000 pounds per square inch gauge, and
a charge rate of said fluid such that the weight hourly space velocity is in the range of from about 0.01 hour⁻¹ to about 1000 hour⁻¹.

51. A process according to claim 45 wherein said contacting of said catalyst composition and said fluid is conducted in the presence of a solvent selected from the group consisting of cyclopentane, cyclohexane, cycloheptane, cyclooctane, and the like and combinations thereof.

52. A process according to claim 51 wherein a weight ratio of said solvent to catalyst composition is in the range of from about 400:1 to about 20:1.

53. A process according to claim 45 wherein said process of preparing a high molecular weight hydrocarbon is conducted in a slurry phase reactor.

54. A process of preparing a catalyst composition comprising contacting a ruthenium component and an oxidizing agent to provide a ruthenium tetraoxide and further wherein said process further comprises depositing under a depositing condition said ruthenium tetraoxide on, in, or with a support component.

55. A process according to claim 54 wherein said ruthenium component is selected from the group consisting of ruthenium bromide, ruthenium bromide hydrate, ruthenium chloride, ruthenium chloride hydrate, ruthenium iodide, ruthenium nitrosyl nitrate, ruthenium oxide, ruthenium oxide hydrate, and combinations thereof.

56. A process according to claim 54 wherein said ruthenium component comprises ruthenium chloride, ruthenium chloride hydrate, or ruthenium nitrosyl nitrate.

57. A process according to claim 54 wherein said oxidizing agent is selected from the group consisting of potassium meta-periodate, periodic acid, potassium permanganate, perchloric acid, potassium chromate, chromic acid, and combinations thereof.

58. A process according to claim 57 wherein said oxidizing agent is potassium meta-periodate.

59. A process according to claim 54 wherein a weight ratio of said ruthenium component to said oxidizing agent is in a range of from about 0.5:1 to about 10:1.

60. A process according to claim 54 wherein an amount of said ruthenium component is such as to provide a concentration of ruthenium in said catalyst composition in the range of from about 0.5 weight percent to about 10 weight percent based on the total weight of said catalyst composition.

61. A process according to claim 54 further comprises heating under a heating condition comprising:
a temperature in the range of from about 20° C. to about 200° C.,
a pressure in the range of from about 0 pounds per square inch absolute to about 50 pounds per square inch absolute, and
a time period in the range of from about 0.1 hour to about 10 hours.

62. A process according to claim 54 wherein said depositing condition comprises:
a temperature in the range of from about 20° C. to about 200° C.,
a pressure in the range of from about 0 pounds per square inch absolute to about 50 pounds per square inch absolute, and
a time period in the range of from about 0.1 hour to about 10 hours.

63. A process according to claim 62 wherein said depositing condition further comprises the use of a carrier gas selected from the group consisting of nitrogen, argon, and combinations thereof.

64. A process according to claim 54 wherein said process of preparing said catalyst composition can be conducted in an apparatus selected from the group consisting of hot wall reactors, cold wall reactors, radiation beam assisted reactors, plasma assisted reactors, and combinations thereof.

65. A process according to claim 54 wherein said support component is selected from the group consisting of oxides of the metals of Groups II, III, IV, V, and VI A of the Periodic Table of the Elements, and combinations thereof.

66. A process according to claim 65 wherein said support component is selected from the group consisting of the oxides of the metals of Groups II, III B and IV B of the Periodic Table of the Elements, and combinations thereof.

67. A process according to claim 66 wherein said support component is selected from the group consisting of alumina, boria, zinc oxide, magnesia, calcium oxide, strontium oxide, barium oxide, titania, zirconia, vanadia, and combinations thereof.

68. A process according to claim 67 wherein said support component is selected from the group consisting of silicon dioxide, zirconyl phosphate, and combinations thereof.

69. A process according to claim 54 further comprises treating under a treating condition comprising:
a temperature in the range of from about 100° C. to about 500° C.,
a pressure in the range of from about 0 pounds per square inch absolute to about 100 pounds per square inch absolute,
a time period in the range of from about 0.1 hour to about 20 hours, and
further wherein said treating condition comprises an atmosphere selected from the group consisting of an oxygen-containing atmosphere, nitrogen, argon, and combinations thereof.

70. A process according to claim 54 further comprises activating under an activating condition comprising:
a temperature in the range of from about 50° C. to about 500° C.,
a pressure in the range of from about 0 pounds per square inch absolute to about 750 pounds per square inch absolute,
a time period in the range of from about 0.1 hour to about 30 hours, and
further wherein said activating condition comprises an atmosphere selected from the group consisting of hydrogen, carbon monoxide, synthesis gas, other reducing gases, and combinations thereof.

71. A process according to claim 68 wherein said zirconyl phosphate is prepared by a process of preparing comprising contacting a zirconyl salt and a water-soluble acidic phosphorous compound.

72. A process according to claim 71 wherein said water-soluble acidic phosphorous compound is selected from the group consisting of phosphoric acid, phosphorous acid, and combinations thereof.

73. A process according to claim 72 wherein said water-soluble acidic phosphorous compound is phosphoric acid.

74. A process according to claim 71 wherein a weight ratio of said zirconyl salt to said water-soluble acidic phosphorous compound is in the range of from about 0.5:1 to about 10:1.

75. A process according to claim 71 wherein said process of preparing said zirconyl phosphate further comprises contacting said zirconyl salt and water-soluble acidic phosphorous compound with a hydroxide component under a precipitating condition to provide for an increase of pH and for precipitating said zirconyl phosphate.

76. A process according to claim 75 wherein said precipitating condition comprises:
a temperature in the range of from about 20° C. to about 90° C.,
a pressure in the range of from about 0 pounds per square inch absolute to about 100 pounds per square inch absolute, and
a time period in the range of from about 0.1 hour to about 10 hours.

77. A process according to claim 71 wherein said zirconyl salt is selected from the group consisting of zirconyl chloride hydrate, zirconyl chloride octahydrate, zirconyl nitrate hydrate, zirconyl nitrate, zirconyl perchlorate octahydrate, and combinations thereof.

78. A process according to claim 71 wherein said zirconyl salt comprises zirconyl nitrate hydrate.

79. A process according to claim 75 wherein said hydroxide component is selected from the group consisting of ammonium hydroxide, tetramethyl ammonium hydroxide, tetramethyl ammonium bromide, tetraethyl ammonium bromide, lithium hydroxide, sodium hydroxide, sodium hydrosulfide, potassium hydroxide, calcium hydroxide, magnesium hydroxide, sodium bicarbonate, sodium carbonate, sodium oxide, sodium sulfate, magnesium oxide, calcium oxide, calcium carbonate, sodium phenoxide, barium phenoxide, calcium phenoxide, and combinations thereof.

80. A process according to claim 75 wherein said hydroxide component comprises ammonium hydroxide.

81. A process according to claim 75 wherein a weight ratio of said hydroxide component to said zirconyl salt and water-soluble acidic phosphorous compound is in the range of from about 0.01:1 to about 1:1.

82. A process according to claim 75 wherein said process of preparing said zirconyl phosphate further comprises contacting said zirconyl phosphate with an aqueous solution.

83. A process according to claim 75 wherein said process of preparing said zirconyl phosphate further comprises contacting with an alcohol selected from the group consisting of methyl alcohol, ethyl alcohol, isopropyl alcohol, butyl alcohol, and combinations thereof.

84. A process according to claim 75 wherein said process of preparing said zirconyl phosphate further comprises drying under a drying condition comprising:
a temperature in the range of from about 20° C. to about 200° C.,
a pressure in the range of from about 0 pounds per square inch absolute to about 200 pounds per square inch absolute,
a time period in the range of from about 0.5 hour to about 10 hours, and
further wherein said drying condition comprises an atmosphere comprising air.

85. A process according to claim 75 wherein said process of preparing said zirconyl phosphate further comprises calcining under a calcining condition comprising:
a temperature in the range of from about 250° C. to about 1000° C.,
a pressure in the range of from about 0 pounds per square inch absolute to about 750 pounds per square inch absolute,
a time period in the range of from about 0.5 hour to about 30 hours, and
further wherein said calcining condition comprises an atmosphere comprising air.

86. A process according to claim 75 wherein said process of preparing said zirconyl phosphate further comprises contacting said zirconyl phosphate with a silica component selected from the group consisting of silica, colloidal silica, silica gel, and combinations thereof.

87. A process according to claim 86 wherein said silica component is silica gel.

88. A process according to claim 86 wherein said contacting said zirconyl phosphate with a silica component occurs during said precipitating of said zirconyl phosphate.

89. A process according to claim 86 wherein a weight ratio of said silica component to said zirconyl phosphate is in the range of from about 0.01:1 to about 1:1.

90. A composition comprising ruthenium and a support component wherein said ruthenium is initially present as ruthenium tetraoxide provided by contacting a ruthenium component and an oxidizing agent to provide said ruthenium tetraoxide that is deposited under a depositing condition on, in, or with said support component.

91. A composition according to claim 90 wherein a concentration of said ruthenium in said catalyst composition is in a range of from about 0.5 weight percent to about 10 weight percent based on the total weight of said catalyst composition.

92. A composition prepared by the process of claim 54.

93. A composition prepared by the process of claim 55.

94. A composition prepared by the process of claim 57.

95. A composition prepared by the process of claim 59.

96. A composition prepared by the process of claim 61.

97. A composition prepared by the process of claim 62.

98. A composition prepared by the process of claim 65.

99. A composition prepared by the process of claim 69.

100. A composition prepared by the process of claim 70.
